(19) United States
(12) Patent Application Publication
(10) Pub. No.: US 2016/0324898 A1
(43) Pub. Date: Nov. 10, 2016

(54) COMPOSITIONS AND METHODS FOR THE TREATMENT OF ALZHEIMER'S DISEASE

(71) Applicant: Stemedica Cell Technologies, Inc., San Diego, CA (US)

(72) Inventors: Tristan Bolmont, Epalinges (CH); Nikolai Tankovich, San Diego, CA (US)

(73) Assignee: Stemedica International, SA, Epalinges (CH)

(21) Appl. No.: 14/997,428

(22) Filed: Jan. 15, 2016

Related U.S. Application Data
(60) Provisional application No. 62/156,396, filed on May 4, 2015.

(51) Int. Cl. A61K 35/28; A61K 38/17; A61K 38/20; A61K 38/45; A61K 38/58; A61K 38/18; A61K 38/28; A61K 9/00; A61K 38/19

(52) U.S. Cl. CPC A61K 35/28; A61K 9/0019; A61K 38/1774; A61K 38/204; A61K 38/2053; A61K 38/2086; A61K 38/20; A61K 38/19; A61K 38/1793; A61K 38/195; A61K 38/177; A61K 38/58; A61K 38/1875; A61K 38/179; A61K 38/1825; A61K 38/1833; A61K 38/1754; A61K 38/28; A61K 38/18; A61K 38/1866; A61K 38/45; C12Y 207/10001

(57) ABSTRACT

Compositions of mesenchymal stem cells and mesenchymal stem cell factors are provided. Methods of using the inventive compositions in the treatment of Alzheimer's disease are contemplated.

### BACKGROUND OF THE INVENTION

1. Field of the Invention

The invention is in the field of treatments for Alzheimer's disease.

2. Description of Related Art

Alzheimer's Disease (AD) is a severe mental health problem that affects an estimated 3-4 million people in the United States (Keefover, 1996, Neurol. Clin. 14:337-351). Many studies have established that AD appears in two distinct forms: an early-onset form and a late-onset, sporadic form. Incidence of the latter form increases with age, and AD is now believed to be the most important single cause of senile dementia in humans. Estimates of the prevalence of late-onset AD based on epidemiological evidence vary, and the incidence of the disease appears to differ with the population examined. However, at least half of the total cases of dementia in the elderly may be attributable to AD (Evans et al., 1989, J. Amer. Med. Assn. 262:2551-2556; Breteler et al., 1992, Epidemiol. Rev. 14:59-82). Thus, AD is a significant mental health concern, which is likely to increase in importance with the continued aging of the population.

The deposition of extracellular amyloid plaques in the brain is a major neuropathological hallmark in AD, first reported by Alois Alzheimer in 1906. These amyloid plaques are primarily composed of aggregated Abeta peptides (Haass and Selkoe, Nature 2007; 8:656-67) generated by the sequential cleavage of amyloid precursor protein (“APP”) via β and γ-secretases. APP, particularly in its oligomerized forms, is toxic to neurons and the subsequent formation of Abeta amyloid deposits is believed to be an early and critical event in the pathogenic cascade leading to cell death and dementia in AD. Therapies that reduce Abeta levels in the brain may alleviate cognitive dysfunction and block further synaptic loss, axon degeneration, and neuronal cell death. Abeta can be transported actively across the blood-brain barrier (Deane et al., Stroke 2004; 35(Suppl I):2628-31). In murine models of AD, systemic delivery of antibodies to Abeta increases Abeta levels in plasma while reducing levels in the central nervous system (CNS) through several proposed mechanisms, including dissolution of brain Abeta plaque, phagocytic removal of opsonized Abeta, and finally via efflux of Abeta from the brain as a result of an equilibrium shift of Abeta resulting from circulating antibodies (Morgan, Neurodegener. Dis. 2005; 2:261-6).

### BRIEF SUMMARY OF THE INVENTION

An object of the invention is to provide compositions of mesenchymal stem cells and mesenchymal stem cell factors for the treatment of Alzheimer's disease.

A further object of the invention is to provide a method for treating Alzheimer's disease in a subject in need thereof comprising administering to the subject a composition comprising at least one stem cell factor.

A method for treating Alzheimer's disease in a subject in need thereof comprising administering to the subject mesenchymal stem cells, and a composition comprising at least one stem cell factor, wherein the at least one stem cell factor is optionally obtained from mesenchymal stem cells.

A kit comprising isolated mesenchymal stem cells; and a composition comprising at least one isolated stem cell factor, wherein the at least one isolated stem cell factor is optionally obtained from mesenchymal stem cells.

### BRIEF DESCRIPTION OF THE SEVERAL VIEWS OF THE DRAWINGS

FIG. 1. Decreased soluble cerebral Aβ42 levels following a single intravenous hMSC injection. Brain protein extracts were collected one week post-delivery of a single hMSC injection and analyzed by ELISA. Reduction of soluble Aβ42 was observed in brains of both young and aged transgenic mice following a single hMSC injection (−28% and −31%, respectively) as compared to control mice treated with LRS.

FIG. 2. Upregulation of Aβ-degrading enzymes in APPPS1 mice following a single intravenous hMSC delivery. Purified protein fractions of brains extracts were analyzed by western blot for several Aβ-degrading enzymes, i.e. Insulin degrading enzyme (IDE), Endothelin-converting enzyme (ECE) and Neprilysin degrading enzyme (NPE). The western blot analysis also includes the tubulin as control. Interestingly, a single hMSC injection was sufficient to increase the level of all three Aβ-degrading enzymes. In young hMSC-treated mice (A), an increase of 40% in IDE signal compared to LRS-treated was observed. In aged hMSC-treated mice (B), a 94% increase in NPE signal was observed compared to control mice and the IDE signal was also increased. Surprisingly, the level of ECE was dramatically increased in both young and aged mice (99% for both groups) following a single injection of hMSC compared to the LRS-treated control mice.

FIG. 3. Modulation of cytokines levels following a single hMSC injection. A large panel of cytokines was analyzed by Mesoscale, including IFNγ, diverse Interleukins (IL1β, IL2, IL4, IL5, IL6, IL10 and IL-12p70), KC/GRO and TNFα. Interestingly, the levels of pro-inflammatory cytokines were significantly reduced following single injection of hMSC. For instance, TNFα levels were markedly decreased in both young and aged hMSC-treated mice compared to control LRS-treated mice (−42% and −45% respectively). In young mice, the levels of IL-12p70, another pro-inflammatory cytokine, were decreased by 53% in hMSC-treated mice compared to control mice. In aged mice, IL-10 levels were decreased by 54% in hMSC-treated mice compared to age-matched, LRS-treated mice. Interestingly, in aged mice, the levels of KC/GRO (CXCL1) was significantly increased.

FIG. 4. Impact of repeated hMSC treatment on Aβ immunoreactivity in APPPS1 transgenic mice. (A) Representative 6E10-stained brain sections encompassing the cortex and hippocampus of a young APPPS1 mouse injected with control LRS, in comparison to a young APPPS1 mouse repeatedly treated with hMSC. Quantification of amyloid load on 6E10-stained sections demonstrates a significant reduction of cerebral Aβ immunoreactivity following hMSC treatment. Young APPPS1 mice treated with hMSC showed a robust decease in amyloid load for both the cortex (−36%, p<0.001) and hippocampus (−50%, p<0.001). (B) Representative brain sections of an aged APPPS1 mouse injected with control LRS, in comparison to an aged APPPS1 mouse repeatedly treated with hMSC. Quantification demonstrates a significant reduction of cerebral Aβ immunoreactivity following hMSC treatment in both the cortex (−36%, p<0.001) and hippocampus (−50%, p<0.001).

FIG. 5. Decreased neuroinflammation following intravenous hMSC treatment in APPPS1 transgenic mice. (A) Compared to control brains treated with LRS, microglial activation was decreased in brains of young transgenic mice treated with hMSC, as demonstrated by quantitative image analysis of Iba-1 immunoreactivity revealing a −39% in microglial immunoreactivity in young APPPS1 mice (p<0.001). (B) Quantitative image analysis of Iba-1 immunoreactivity reveals a −35% in microglial immunoreactivity in aged APPPS1 mice (p<0.001).

FIG. 6. Biodistribution of hMSC in APPPS1 mice following intravenous delivery. (A) At 1 hour after the intravenous hMSC injection, biodistribution analysis revealed that hMSC are detected in APPPS1 brains (MSC), whereas LRS injection yielded no detectable signal (LRS) in APPPS1 brains. The amount of hMSC detected in brains subsequently decreased at 1 day post-delivery. The cerebral levels of hMSC were below detection level at 1 week post-delivery. In comparison at 1 hour post-delivery, the amount of cells detected in the heart (C), spleen (E) and kidney (F) were approximately 7 times higher. Compared to the brain, the amount of hMSC detected in liver (B) were approximately 10 times higher and in the lung (D) 20 times higher. At 1 hour post-delivery, approximately 30% of hMSC relocated to the brain parenchyma and 70% were located within cerebral blood vessels.

FIG. 7. Impact of repeated intranasal application of soluble hMSC factors (SCF) on cerebral Aβ amyloidosis. (A) Representative 6E10-stained brain sections encompassing the cortex and hippocampus of an aged APPPS1 mouse that received the repeated SCF intranasal administration, as compared to (B) LRS application in a control APPPS1 mouse. (B) Quantification of amyloid load on 6E10-stained sections demonstrates a significant reduction of cerebral Aβ immunoreactivity following SCF intranasal applications (−25%, p<0.01).

FIG. 8. Impact of repeated hMSC treatment on congophilic amyloid pathology in aged APPPS1 transgenic model. (A) Representative Thioflavin T-stained brain sections encompassing the cortex and hippocampus of an aged APPPS1 mouse injected with control LRS, in comparison to an aged APPPS1 mouse repeatedly treated with hMSC. (B) Quantification of compact amyloid load on Thioflavin T-stained brain sections revealed a significant reduction in aged APPPS1 mice following hMSC treatment (−30%, p<0.0001), as compared to aged-matched control APPPS1 mice intravenously injected with LRS. Aged APPPS1 mice treated with hMSC exhibit a robust decease in amyloid load for both the cortex (−25%, p<0.00001) and hippocampus (−36%, p<0.0001).

FIG. 9. No increase in cerebral amyloid angiopathy in APPPS1 mice following intravenous hMSC delivery. (A) Representative Thioflavin-stained brain sections of a young APPPS1 mouse injected with control LRS, in comparison to a young APPPS1 mouse repeatedly treated with hMSC. (B) Representative Thioflavin-stained brain sections of an aged APPPS1 mouse injected with control LRS, in comparison to an aged APPPS1 mouse repeatedly treated with hMSC. (C) As positive control, brain sections from another APP transgenic mouse model that develops robust vascular amyloid pathology were used.

FIG. 10. Absence of microhemorrhages in APPPS1 mice following intravenous hMSC delivery. The numbers of microhemorrhages following hMSC delivery was evaluated by hemosiderin staining in APPPS1 brains. (A) Representative hemosiderin-stained brain sections of an aged APPPS1 mouse injected with control LRS, in comparison to an aged APPPS1 mouse repeatedly treated with hMSC. As positive control, tissue sections from human liver with extensive iron deposition were used, which gives this characteristic blue hemosiderin-positive profile (B). Statistics demonstrate that treatment with hMSC did not induce microhemorrhages in young or aged APPPS1 mice as compared to control LRS treatment (C).

FIG. 11. Biodistribution study in APPPS1 Alzheimer mice versus healthy brains at 1 hour after intravenous injection. Aged, 12.5 month-old APPPS1 mice (n=5) and wild-type mice (n=5) received an intravenous hMSC delivery (500,000 cells resuspended in 100 μl of LRS). As controls, age-matched uninjected APPPS1 mice (n=4) as well as APPPS1 received an intravenous delivery of LRS (100 μl, n=4).

FIG. 12. Concentration of selected soluble factors secreted by hMSC. Cells were expanded at 5% oxygen. Serum-free cell-conditioned medium was harvested, the medium was concentrated ˜50 times, mixed with sucrose, and then preserved by vacuum foam drying technique. Two ml of water was added to a bottle containing dried sucrose-protein foam. Concentrations of solubilized proteins are given below for one bottle. 37 out of 120 proteins were found in quantities greater than 0.5 ng per bottle.

### Definitions

As used herein, the singular form “a”, “an” and “the” include plural references unless the context clearly dictates otherwise. For example, the term “a compound” or “at least one compound” may include a plurality of compounds, including mixtures thereof.

The term “about” or “approximately” as used herein usually means within 20%, more preferably within 10%, and most preferably still within 5% of a given value or range. Alternatively, especially in biological systems, the term “about” means within about a log (i.e., an order of magnitude) preferably within a factor of two of a given value.

The phrase “Alzheimer's disease” as used herein refers to a progressive disease of the human central nervous system. It is manifested by dementia typically in the elderly, by disorientation, loss of memory, difficulty with language, calculation, or visual- spatial skills, and by psychiatric manifestations. It is associated with degenerating neurons in several regions of the brain. Histologically, the disease is characterized by neuritic plaques, found primarily in the association cortex, limbic system and basal ganglia. The phrase “Alzheimer's disease” as used herein is intended to encompass all types of Alzheimer's disease, including sporadic and familial AD, as well as late onset and early onset AD.

The phrase “early Alzheimer's disease” as used herein includes patients with mild cognitive impairment, such as a memory deficit, due to Alzheimer's disease and subjects having Alzheimer's disease biomarkers, for example amyloid positive patients.

The phrase “mild Alzheimer's disease” as used herein refers to a stage of Alzheimer's disease characterized by an MMSE score of 20 to 26.

The phrase “mild to moderate Alzheimer's disease” as used herein encompasses both mild and moderate Alzheimer's disease characterized by an MMSE score of 18 to 26.

The phrase “moderate Alzheimer's Disease” refers to a stage of Alzheimer's disease characterized by an MMSE score of 18 to 19.

The phrase “severe or late-stage Alzheimer's disease” as used herein refers to very severe cognitive decline when individuals lose the ability to respond to their environment, the ability to speak and, ultimately, the ability to control movement.

The term “late-onset Alzheimer's disease” as used herein refers to Alzheimer's disease which has a time of onset after the subject reaches 40 years of age.

As used herein, Alzheimer's disease is “alleviated” if the severity of a symptom of the AD, the frequency with which such a symptom is experienced by a patient, or both, are reduced.

The terms “amyloid beta,” “beta-amyloid,” “Abeta,” “amyloidβ,” and “Aβ”, are used interchangeably herein to refer to the fragment of amyloid precursor protein (“APP”) that is produced upon β-secretase 1 (“BACE1”) cleavage of APP, as well as modifications, fragments and any functional equivalents thereof, including, but not limited to, Aβ1-40, and Aβ1-42. Aβ is known to exist in monomeric form, as well as to associate to form oligomers and fibril structures, which may be found as constituent members of amyloid plaque. The structure and sequences of such Aβ peptides are well known to one of ordinary skill in the art and methods of producing said peptides or of extracting them from brain and other tissues are described, for example, in Glenner and Wong, Biochem Biophys Res. Comm. 129: 885-890 (1984). Moreover, various forms of Aβ peptides are commercially available.

The term “amyloidosis,” as used herein refers to a group of diseases and disorders caused by or associated with amyloid or amyloid-like proteins and includes, but is not limited to, diseases and disorders caused by the presence or activity of amyloid-like proteins in monomeric, fibril, or polymeric state, or any combination of the three, including by amyloid plaques. Such diseases include, but are not limited to, secondary amyloidosis and age-related amyloidosis, such as diseases including, but not limited to, neurological disorders such as Alzheimer's Disease, diseases or conditions characterized by a loss of cognitive memory capacity such as, for example, mild cognitive impairment (MCI), Lewy body dementia, Down's syndrome, hereditary cerebral hemorrhage with amyloidosis (Dutch type), the Guam Parkinson-Demential complex and other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), inclusion-body myositis (IBM), adult onset diabetes, endocrine tumor and senile cardiac amyloidosis, and various eye diseases including macular degeneration, drusen-related optic neuropathy, glaucoma, and cataract due to beta-amyloid deposition.

The phrase “cerebral amyloidosis” as used herein refers to a pathological condition of small cerebral vessels characterized by deposits of amyloid in the vessel walls which may lead to infarcts or hemorrhage.

The term “cerebral macrohemorrhage” refers to an intracranial hemorrhage, or bleeding in the brain, of an area that is more than about 1 cm in diameter. Cerebral macrohemorrhage is detectable by, e.g., brain MRI, including but not limited to T2*-weighted GRE MRI, and can be asymptomatic (“asymptomatic macrohemorrhage”) or associated with symptoms such as transient or permanent focal motor or sensory impairment, ataxia, aphasia, and dysarthria (“symptomatic macrohemorrhage”) (see, e.g., Chalela J A, Gomes J. Expert Rev. Neurother. 2004 4:267, 2004 and Sperling et al. Alzheimer's & Dementia, 7:367, 2011).

The term “differentiation” as used herein refers to the biological process by which a less specialized cell becomes a more specialized cell type. For example, during embryonic development, pluripotent embryonic stem cells “differentiate” to form multipotent mesenchymal, ectodermal and endodermal stem cells, each of which are limited to a specific developmental pathway (i.e. range of tissues).

“Differentiation potential,” “cell potential,” “plasticity” and “potential” are used interchangeably herein to refer to the ability of a stem cell to differentiate into one or more specialized cell types.

As used herein, the phrase “effective amount” or “effective dose” of an agent refers to an amount or dose effective, for periods of time necessary, to achieve the desired result. For example, a “therapeutically effective amount” is an amount effective, for periods of time necessary, to treat the indicated disease, condition, clinical pathology, or symptom, i.e., to modify the course of progression of Alzheimer's disease and/or to alleviate and/or prevent one or more symptoms of Alzheimer's disease.

The term “isolated” as used herein refers to the removal of a substance from its natural environment such that it is free or substantially free of other substances, such as substances with which it is normally associated with in nature. An isolated substance can be about 100%, 99%, 98%, 97%, 96%, 95%, 90%, 85%, 80%, 75%, 70%, 65% or 60% free of other substances, as well as any intervening value.

The phrases “low oxygen conditions,” “low oxygen,” “reduced oxygen tension,” and “hypoxia” as used herein refer to any oxygen concentration that is less than atmospheric oxygen. Low oxygen conditions include, but are not limited to, less than about 20%, 19%, 18%, 17%, 16%, 15%, 14%, 13%, 12%, 11%, 10%, 9%, 8%, 7%, 6%, 5%, 4%, 3%, 2%, or 1% oxygen.

The phrase “mesenchymal stem cell” as used herein refers to a stem cell that is not terminally differentiated, which can divide to yield cells that are either stem cells, or which, irreversibly differentiate to give rise to cells of a mesenchymal cell lineage. The mesenchymal stem cells can be multipotent stem cells or progenitor cells.

As used herein, the phrase “mesenchymal stem cell factor,” or “MSCF,” refers to any substance or substances produced through the metabolic activity of a mesenchymal stem cell. MSCF include, but are not limited to, cytokines, chemokines, peptides, proteins, amino acids, polynucleotides (i.e. RNA or DNA), and combinations thereof. MCSF can be secreted proteins and/or intracellular proteins.

The term “multipotent.” or “multipotency” as used herein refers to the ability of a stem cell to form more than one cell type belonging to a single germ lineage (e.g. the endoderm or ectoderm or mesoderm). For example, a cell which has the ability to form chondrocytes, adipocytes and osteocytes is a multipotent mesenchymal cell.

The term “normoxic” as used herein refers to atmospheric oxygen levels. Normoxic oxygen concentration can be 20% oxygen or above.

The phrase “pharmaceutically acceptable carrier” as used herein refers to an ingredient in a pharmaceutical formulation, other than an active ingredient, which is nontoxic to a subject. A pharmaceutically acceptable carrier includes, but is not limited to, a buffer, excipient, stabilizer, and/or preservative.

The phrase “precursor cell,” “tissue precursor cell,” or “progenitor cell” as used herein refers to an undifferentiated cell that is committed, dedicated or fated to a specific developmental pathway. Precursor cells have limited proliferative ability. For example, a “neural precursor cell” is a cell that is dedicated to the development of a neuron, glial cell or astrocyte.

The phrase “stem cell” as used herein refers to an undifferentiated cell which has the ability to both self-renew (through mitotic cell division) and undergo differentiation to form a more specialized cell. Stem cells have varying degrees of potency. Precursor cells and progenitor cells are non-limiting examples of stem cells.

The term “subject” as used herein typically refers to a human. In certain embodiments, a subject is a non-human mammal. Exemplary non-human mammals include laboratory, domestic, pet, sport, and stock animals, e.g., mice, cats, dogs, horses, and cows. Typically, the subject is eligible for treatment, e.g., displays one or more indicia of disease being treated. Generally, such subject or patient is eligible for treatment Alzheimer's disease. In one embodiment, such eligible subject or patient is one that is experiencing or has experienced one or more signs, symptoms, or other indicators of Alzheimer's disease or has been diagnosed with Alzheimer's disease, whether, for example, newly diagnosed, previously diagnosed for developing Alzheimer's disease. Diagnosis of Alzheimer's disease may be made based on clinical history, clinical examination, and established imaging modalities. A “patient” or “subject” herein includes any single human subject eligible for treatment who is experiencing or has experienced one or more signs, symptoms, or other indicators of Alzheimer's disease. Intended to be included as a subject are any subjects involved in clinical research trials, or subjects involved in epidemiological studies, or subjects once used as controls.

As used herein, the term “treatment” (and grammatical variations thereof such as “treat” or “treating”) refers to clinical intervention comprising the method(s) of the present invention in an attempt to alter the natural course of Alzheimer's disease in the subject being treated. Desirable effects of treatment include, but are not limited to, alleviation or amelioration of one or more symptoms, diminishment of or delay in the appearance of or worsening of any direct or indirect pathological consequences of the disease, decrease of the rate of disease progression, and amelioration or palliation of the disease state.

### DETAILED DESCRIPTION OF THE INVENTION

The invention generally relates to methods and compositions for the treatment of Alzheimer's disease. More particularly, the invention relates to compositions of mesenchymal stem cells and/or mesenchymal stem cell factors and methods for their use in the treatment of Alzheimer's disease.

In some embodiments, the invention provides a method for treating Alzheimer's disease in a subject comprising administering to the subject a composition comprising at least one MSCF. MSCF for use with the invention may be obtained (e.g. collected) from any mesenchymal stem cell capable of providing a MCSF having a therapeutic effect in the treatment of Alzheimer's disease. MCSF can obtained from mesenchymal stem cells having varying degrees of potency, including multipotent mesenchymal stem cells and precursor cells of mesenchymal lineage. MCSF can be obtained from a population of mesenchymal stem cells having the same differentiation potential, or a mixed population of mesenchymal stem cells having varying degrees of differentiation potential. MCSF can be obtained from a population of multipotent mesenchymal stem cells, progenitor cells of mesenchymal lineage, or a combination thereof. MCSF can be obtained from a population of isolated multipotent mesenchymal stem cells, a population of isolated progenitor cells of mesenchymal lineage, or a combination thereof. Such isolated populations of cells can be, for example, cells obtained from the expansion of a clonal cell, or cells purified from a mixed population of cells, such as by FACS.

MSCF can be obtained from mesenchymal stem cells disclosed herein which are grown under in vitro conditions, a population of mesenchymal stem cells that are obtained from the tissue of a donor (e.g. a primary cell culture or tissue explant), or a combination thereof. Mesenchymal stem cells for producing MSCF can be exposed to normoxic conditions, low oxygen conditions, or a combination thereof. Mesenchymal stem cells for producing MCSF can be cultured for one or more passages under low oxygen conditions, under normoxic conditions, or a combination of low oxygen conditions and normoxic conditions. Suitable processes, reagents and equipment for culturing mesenchymal stem cells under low oxygen conditions are disclosed in the following references, the disclosures of which are incorporated herein by reference for all purposes: U.S. Pat. No. 6,759,242; U.S. Pat. No. 6,846,641; U.S. Pat. No. 6,610,540; J. Cereb. Blood Flow Metab. 2008 September 28(9):1530-42; Stem Cells. 2008 May 26(5):1325-36; Exp Neurol. 2008 April 210(2):656-70; Mol. Cell. Neurosci. (2007), doi: 10.1016/j .mcn.2007.04.003; Experimental Neurology 170, 317-325 (2001); and Neurosignals 2006-07, 15:259-265. Although these references disclose particular procedures and reagents, any low oxygen culture condition capable of expanding mesenchymal stem cells may be used. Mesenchymal stem cells for producing MCSF can be exposed to low oxygen conditions, normoxic conditions, or a combination of low oxygen conditions without expanding the mesenchymal stem cells. Such exposure can occur, for example, by incubating the mesenchymal stem cells for a period of time that is less than an amount of time required for the cells to divide. Without limiting the possible embodiments of the invention, low oxygen conditions as disclosed herein can be 5% oxygen.

Mesenchymal stem cells for providing MCSF can be obtained from any source of mesenchymal stem cells capable of providing MCSF that produce a therapeutic effect in the treatment of Alzheimer's disease when administered according to the methods disclosed herein. Mesenchymal stem cells for providing MCSF for use with the invention include, but are not limited to, MSC obtained from prenatal sources, postnatal sources, and combinations thereof. Tissues for deriving a suitable source of mesenchymal stem cells for producing MSCF include, but are not limited to, bone marrow, dermis, periosteum, synovium, peripheral blood, skin, hair root (e.g. dermal papilla), muscle, uterine endometrium, adipose, placenta, menstrual discharge, chorionic villus, amniotic fluid and umbilical cord blood. Mesenchymal stem cells for producing MCSF may be derived from these sources individually, or the sources may be combined (before or after enrichment) to produce a mixed population of mesenchymal stem cells from different tissue sources. Mesenchymal stem cells for producing MSCF can be obtained from adult tissues, fetal tissues, differentiated from embryonic or induced pluripotent stem cells, or a combination thereof.

MCSF can be obtained from mesenchymal stem cells that have been expanded under in vitro conditions, mesenchymal stem cells obtained from a tissue explant, or a combination thereof. MCSF can be obtained from mesenchymal stem cells purified from a cultured population of mesenchymal stem cells, or purified from a tissue explant.

MCSF can be obtained from the mesenchymal stem cells disclosed in the following references, the disclosures of which are incorporated by reference herein in their entirety for all purposes: U.S. Pat. No. 5,215,927; U.S. Pat. No. 5,225,353; U.S. Pat. No. 5,262,334; U.S. Pat. No. 5,240,856; U.S. Pat. No. 5,486,359; U.S. Pat. No. 5,759,793; U.S. Pat. No. 5,827,735; U.S. Pat. No. 5,811,094; U.S. Pat. No. 5,736,396; U.S. Pat. No. 5,837,539; U.S. Pat. No. 5,837,670; U.S. Pat. No. 5,827,740; U.S. Pat. No. 6,087,113; U.S. Pat. No. 6,387,367; U.S. Pat. No. 7,060,494; Jaiswal, N., et al., J. Cell Biochem. (1997) 64(2): 295 312: Cassiede P., et al., J. Bone Miner. Res. (1996) 11(9): 1264 1273; Johnstone, B., et al., (1998) 238(1): 265 272; Yoo, et al., J. Bone Joint Sure. Am. (1998) 80(12): 1745 1757; Gronthos, S., Blood (1994) 84(12): 41644173; Basch, et al., J. Immunol. Methods (1983) 56: 269; Wysocki and Sato, Proc. Natl. Acad. Sci. (USA) (1978) 75: 2844; and Makino, S., et al., J. Clin. Invest. (1999) 103(5): 697 705.

Mesenchymal stem cells for providing MCSF may be obtained from donor sources that are allogeneic, syngeneic, or xenogeneic with respect to the subject that is to be treated with the MSCF. In some embodiments, the MSCF are obtained from a human donor source that is allogeneic with respect to the subject to be treated. The MSCF from allogeneic donors of the mesenchymal stem cells can be HLA matched with the subject that is to be treated.

MSCF can be obtained from homogenates, including homogenates of mesenchymal stem cells grown in culture, homogenates of mesenchymal stem cells purified from a tissue source, or homogenates of tissues containing mesenchymal stem cells. MSCF can be obtained from mesenchymal stem cell conditioned medium. MSCF can be obtained from the foregoing homogenates and mesenchymal stem cell conditioned medium. The mesenchymal stem cell conditioned medium can be medium that was used to grow mesenchymal stem cells under low oxygen conditions, normoxic oxygen conditions, or a combination thereof. The mesenchymal stem cell conditioned medium can be medium that was used to grow mesenchymal stem cells under low oxygen, low serum conditions. As used herein, the term “low serum,” or “low serum conditions,” refers to the culture of cells in a culture medium that comprises less than about 5% serum. Low serum conditions include growing cells in a culture medium having a concentration of about 0.1% and 0.2% serum. The serum can be obtained from sources including, but not limited to, human, bovine, goat, pig, horse, rabbit, rat, and combinations thereof.

Specific examples of MSCF include, but are not limited to: BLC, Eotaxin-1, Eotaxin-2, G-CSF, GM-CSF, I-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Ax1, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP. The methods and compositions disclosed herein can be practiced with one or more of the MSCF disclosed in this paragraph. The methods and compositions disclosed herein can be practiced with all the MSCF disclosed in this paragraph. The invention can be practiced with any combination of the MSCF disclosed in this paragraph, wherein one or more of the foregoing MSCF are specifically excluded. MSCF can comprise functional fragments, analogs or derivatives of the MSCF disclosed herein.

In one non-limiting embodiment of the invention, the invention is practiced with MSCF comprising ICAM-1, IL-6, IL-8, IL-15, IL-16, OPN, TIMP-1, TIMP-2, TNF RI, PF4, MCF R, BMP-5, EGF R, bFGF, FGF-4, FGF-7, HGF, IGFBP-1, IGFBP-2, IGFBP-3, IGFBP-4, IGFBP-6, OPG, Insulin, IGF-I, SCF, MCSF, VEGF, Ax1, CXCL16, ENA-78 (CXCL5), GRO, IL-29, MCP-1, MDC, MIF, and GCP-2.

It will be appreciated that the invention can be practiced with one or more of the following cellular factors which are obtained from a source (e.g. cell) other than a mesenchymal stem cell: BLC, Eotaxin-1, Eotaxin-2, G-CSF, GM-CSF, I-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Ax1, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP. Sources other than mesenchymal stem cells for the foregoing cellular factors include non-mesenchymal stem cells grown in culture (e.g. clonal cell lines), recombinant sources, or the isolation of the cellular factors from tissue.

MSCF can be obtained from recombinant sources by transfecting one or more cells with one or more polynucleotides that encode the MSCF disclosed herein. Mesenchymal stem cells may also be modified to achieve increased expression of one or more of the MSCF disclosed herein by transfecting mesenchymal stem cells with a polynucleotide that encodes the MSCF for which increased expression is desired.

The methods and compositions disclosed herein can be practiced with lyophilized MSCF. MSCF may be lyophilized by any means suitable for producing a composition of MSCF that produces a therapeutic effect when administered to a subject in the treatment of Alzheimer's disease. MSCF may be vaporized according to the methods disclosed in U.S. Patent Application Publication Numbers 2008/0229609 and 2010/0120014, the entire contents of which are incorporated by reference herein in their entirety for all purposes.

MSCF can be formulated with a pharmaceutically acceptable carrier to render a pharmaceutical composition of MSCF suitable for administration to a subject. MSCF can be formulated with a carrier for administration orally, parenterally, sublingually, transdermally, rectally, transmucosally, topically, via inhalation, via buccal administration, intrapleurally, intravenously, intraarterially, intraperitoneally, subcutaneously, intramuscularly, intranasally, intrathecally, intravaginally, retrobulbarly, intraarticularly or a combination thereof. MSCF can suspended in one or more of the pharmaceutically acceptable carriers disclosed in Remington's Pharmaceutical Sciences, 19th Edition, Mack Publishing Co., Easton, Pa. 1995, the entire contents of which are incorporated herein by reference in their entirety for all purposes. MSCF can be combined with one or more artificial pharmaceutically acceptable carriers. In one non-limiting embodiment, lyophilized (e.g. vaporized) MSCF are suspended in one or more of the pharmaceutically acceptable carriers disclosed herein. Such suspended, lyophilized MSCF can be combined with one or more of chitosan or heparin. Without being bound by any particular mechanism or theory, formulating MSCF with chitosan and/or heparin increases the absorption of the MSCF when administered intranasally. MSCF can be administered in the form of a lyophilized (e.g. powdered) preparation. Such preparation can be administered intranasally and may optionally comprise at least one of chitosan and heparin.

In some aspects, the invention provides a method of treating, at least once, Alzheimer's disease comprising administering to a subject in need thereof an amount of MSCF sufficient to treat Alzheimer's disease in the subject. MSCF can be administered to the subject alone, or in combination with the administration of mesenchymal stem cells. MSCF can be administered to the subject before the administration of mesenchymal stem cells to the subject, after the administration of mesenchymal stem cells to the subject, or both before and after the administration of mesenchymal stem cells to the subject. MCSF can be administered after the administration of mesenchymal stem cells to the subject as a means for furthering or maintaining the therapeutic effect of the mesenchymal stem cells in the treatment of Alzheimer's disease. The mesenchymal stem cells for administration to a subject in the treatment of Alzheimer's disease can be the same as those mesenchymal stem cells for obtaining MCSF as disclosed in the present specification. Mesenchymal stem cells can be administered orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, intravaginal, and intraarticular or combinations thereof.

In some embodiments, the invention provides a kit for the treatment of Alzheimer's disease. The kit can comprise one or more isolated MCSF. The kit can comprise one or more isolated MSCF and isolated mesenchymal stem cells. The kit can contain the MSCF and mesenchymal stem cells in one container, or in separate containers. The kits can contain one or more containers of isolated MSCF and one or more containers of isolated mesenchymal stem cells. The MSCF can be derived from mesenchymal stem cell conditioned medium. The MSCF can be derived from mesenchymal stem cell conditioned medium that has been used to grow mesenchymal stem cells under low oxygen, low serum culture conditions. The MSCF can be derived from mesenchymal stem cell conditioned medium that has been used to grow mesenchymal stem cells under low oxygen conditions. The MSCF in the kits can be lyophilized or vaporized as disclosed herein. The kits can comprise one or more pharmaceutical carriers for administering the MSCF and/or mesenchymal stem cells according to the administration routes disclosed herein. The kits can comprise at least one of chitosan and heparin. The chitosan and heparin can be in the form of a powder or solution. The containers for the kit can be vacuum sealed glass vials sealed with a rubber stopper. The containers for the kit can be syringes or other device suitable for intranasal administration.

In one non-limiting embodiment, the invention provides a kit comprising: MSCF derived from mesenchymal stem cell conditioned medium that has been used to grow mesenchymal stem cells under low oxygen, low serum culture conditions, wherein the MSCF of the kit may be in the form of a powder or a solution and optionally comprises at least one of a heparin and chitosan; and a pharmaceutically acceptable carrier suitable for administering the mesenchymal stem cell factors via intranasal administration.

In one non-limiting embodiment, the invention provides a kit comprising at least one isolated MSCF and isolated mesenchymal stem cells, wherein the isolated mesenchymal stem cells are grown under low oxygen conditions, and wherein the at least one isolated MSCF comprises one or more of: BLC, Eotaxin-1, Eotaxin-2, G-CSF, GM-CSF, 1-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Ax1, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP.

In one non-limiting embodiment, the invention provides a kit for treating Alzheimer's disease, wherein the kit comprises isolated MSCF and isolated mesenchymal stem cells, wherein the isolated mesenchymal stem cells are grown under low oxygen conditions, and wherein the at least one MSCF comprises ICAM-1, IL-6, IL-8, IL-15, IL-16, OPN, TIMP-1, TIMP-2, TNF RI, PF4, MCF R, BMP-5, EGF R, bFGF, FGF-4, FGF-7, HGF, IGFBP-1, IGFBP-2, IGFBP-3, IGFBP-4, IGFBP-6, OPG, Insulin, IGF-I, SCF, MCSF, VEGF, Ax1, CXCL16, ENA-78 (CXCL5), GRO, IL-29, MCP-1, MDC, MIF, and GCP-2.

The kits disclosed herein can comprise lyophilized MSCF. The kits disclosed herein can comprise lyophilized or vaporized MSCF combined with one or more of chitosan and heparin. The kits disclosed herein can comprise one lyophilized or vaporized MSCF in a container, and at least one separate container with chitosan and heparin. The kits disclosed herein can contain lyophilized or vaporized MSCF and a pharmaceutically acceptable carrier.

The methods disclosed herein can be used to treat Alzheimer's disease in a subject in need thereof. Subjects can have mild Alzheimer's disease. Subjects can have mild to moderate Alzheimer's disease. Subjects can have moderate Alzheimer's disease. Subjects can have severe or late-stage Alzheimer's disease. Subjects can be newly diagnosed or previously diagnosed as having one of the foregoing stages of Alzheimer's disease. A subject can have in their brain one or more of neuroinflammation, abnormal levels of beta peptide, abnormal levels of soluble beta peptide, abnormal levels of congophilic amyloid beta peptide, and diffuse amyloid beta peptide. As used herein, an abnormal level of one or more of the foregoing parameters is a level that is etiologically advanced over a subject that does not have mild Alzheimer's disease. Subjects can demonstrate cognitive deficits, including but not limited to, memory deficits, confusion, behavioral problems, inability to care for oneself, gradual physical deterioration, or a combination thereof.

Suitable subjects include those who have not previously been diagnosed as afflicted with Alzheimer's disease, those who have previously been determined to be at risk of developing Alzheimer's disease, and those who have been initially diagnosed as being afflicted with Alzheimer's disease where confirming information is desired. Thus it is contemplated that the methods described herein can be used in conjunction with other clinical diagnostic information known or described in the art which are used in evaluation of subjects with Alzheimer's disease or suspected to be at risk for developing such disease. Subjects at risk or suspected of being at risk of developing Alzheimer's disease (i.e. presymptomatic subjects) may be identified by various biomarkers known by those skilled in the art. Suitable biomarkers include, but are not limited to, ratios of Aβ₄₂/Aβ₄₀, LRRK2 gene polymorphisms (e.g. T1602S and T2352) levels of T-tau protein and P-tau protein, ApoE gene variants (e.g. ApoE2, ApoE3 and ApoE4), and combinations thereof. Biomarkers for identifying subjects at risk of developing Alzheimer's disease are disclosed in Chintamaneni et al. “Biomarkers in Alzheimer's Disease: A Review” ISRN Pharmacol. 2012; 2012: 984786, the entire contents of which are incorporated by reference herein in their entirety for all purposes.

The methods disclosed herein can treat Alzheimer's disease by producing therapeutic effects when administered to a subject in need of treatment for Alzheimer's disease. Such therapeutic effects include, but are not limited to, reducing amyloid beta peptides in the brain, reducing amyloid beta peptide plaques in the brain, reducing neuroinflammation in the brain, reducing at least one of congophilic and diffuse amyloid beta peptide in the brain, reducing soluble amyloid beta peptide in the brain, and reducing inflammatory cytokines in the brain. Therapeutic effects can include increasing in the subject at least one of insulin degrading enzyme, neprilysin degrading enzyme, and endothelin converting enzyme-1 compared to a control subject. Therapeutic effects can include reducing the clinical symptoms of Alzheimer's disease, including, but not limited to cognitive deficits, memory deficits, confusion, behavioral problems, inability to care for oneself, gradual physical deterioration, psychosis, or a combination thereof. Therapeutic effects can include postponing the onset of Alzheimer's disease in a subject at risk of developing the disease, or slowing the progression of Alzheimer's disease in a subject with advancing disease. The methods disclosed herein can produce the aforementioned therapeutic effects without increasing cerebral amyloid angiopathy or microhemorrhages in the brain of the subject being treated.

### EXAMPLE 1

### Administration of Mesenchymal Stem Cells

Experimental Study Design for In Vivo Experiments

The following experiment was conducted to evaluate the efficacy of mesenchymal stem cells grown under low oxygen conditions in the treatment of Alzheimer's disease. All animal procedures were performed according to the guidelines of the local authorities and Swiss animal protection law. A group of APPPS1 mice received a single intravenous hMSC injection; another group received a weekly intravenous hMSC injection (500,000 cells/injection) for a total of 10 weeks. One injection consisted of a delivery through the tail vein of 500,000 cells in 100 μl of LRS. The animals were sacrificed one week after the (last) injection. To facilitate the administration procedure, the animals were accommodated into a restraining box and the tail vein revealed by trans-illumination with an optical fiber. For intranasal application, the following procedure was used to deliver the soluble hMSC factors. The animal was restrained by hand without anesthesia, making sure to apply just enough firmness to the skin around the neck to prevent the mouse from turning or twisting out of the restraint, while avoiding to pull the skin so tightly that the animal cannot breathe. For intranasal delivery, using a 10 μl pipette, the required amount of the material to be inhaled was placed at the nares of the animal. The animal was kept restrained on its back until the material disappeared into the nares. Regular observation of the animals during the experiments revealed no overt behavioral change due to the treatments.

Mice

For the in vivo experiments, APPPS1 transgenic mice were obtained from M. Jucker (HIH, Tübingen, Germany) and maintained at the EPFL animal core facility. The mice co-express under the control of the Thy-1 promoter the KM670/671NL Swedish mutation of human amyloid precursor protein (APP) and the L166P mutation of human presenilin 1 (PSI), and usually show the first amyloid plaques in the cortex at an aged of 6-8 weeks. Minimal vascular Aβ amyloidosis is observed and is predominantly restricted to the pial vessels (Radde et al., 2006). APPPS1 mice were generated on a C57BL/6 background and both male and female APPPS1 mice as well as aged-matched control non-transgenic littermates were used. Mice were housed in groups of 5 in pathogen-free conditions until the beginning of the injection experiments, after which they were singly housed.

Preparation of hMSC for Injection

The required number of vials of frozen cells was thawed in a 37° C. water bath (1 vial contains 15×10⁶ cells in 1 ml of freeze media with 10% DMSO). Vials were kept in the water bath until a small (˜2-3 mm) ice crystal remained. The content of the vials (1 ml) was transferred into the 225 ml centrifuge tube. 20 ml of pre-warmed Lactated Ringers Solution (LRS) was gently added (drop wise) and gently mixed. Then, pre-warmed LRS was slowly added to a volume of 180 ml and mixed until homogenous. The tube was centrifuged at 600×g for 5 min at room temperature. The pellet was then decanted in the 225 ml tube down as close to the pellet as possible and the supernatant discarded. Subsequently, 20 ml of pre-warmed LRS was gently added and mixed. Pre-warmed LRS was slowly added to a volume of 180 ml and mixed until homogenous. The tube was centrifuged at 600×g for 5 min at room temperature. The pellet was decanted in the 225 ml tube down as close to the pellet as possible and the supernatant again discarded. Then 20 ml of chilled LRS was slowly added to the 225 ml tube and mixed until homogenous. Additional chilled LRS was slowly added to the 225 ml tube to a volume less than the required cell dose. For cell count, 100 μl was set aside. The cells were counted to determine yield and viability. The required volume of chilled (2-8° C.) LRS was slowly added to a concentration of 5×10⁶ cells/ml based on the cell count.

Histology and Immunohistochemistry

One week after hMSC delivery in the once treated group, or after the last hMSC delivery in the weekly treated group, mice were deeply anesthetized and perfused transcardially with ice-cold PBS (pH 7.4 for two minutes) followed by 4% paraformaldehyde in ice-cold phosphate buffer saline PBS (8 minutes). Brains were removed and postfixed overnight in the same fixative followed by 48 hours incubation in 30% sucrose at the temperature of 4° C. Brains were then frozen in 2-propanol (Merck, Darmstadt, Germany) and subsequently sectioned on a freezing-sliding microtome to collect 25 μm free-floating coronal sections. Sections were immunostained to visualize Aβ deposits using a mouse monoclonal antibody (6E10, 1:500; Covance, Emeryville, Calif.). These anti-Aβ antibodies bind to both diffuse and compact amyloid. Secondary antibodies for peroxidase staining were obtained from Vector Laboratories (ImmPRESS Ig Polymer Detection Kit) and revelation was performed using Vector SG Substrate kit (Vector Laboratories, Burlingame, Calif.). Additionally the dye Thioflavin T (ThT 1%, w/v of 50% Ethanol; Sigma Aldrich, St. Louis, Mo.) was used to specifically assess congophilic amyloid and cerebral amyloid angiopathy. The presence of microhemorrhages was evaluated using hemosiderin histological staining. The slides were then coverslipped using Vectashield Mounting Media (Vector Laboratories, Burlingame, Calif.) with and without DAPI counterstaining to confirm cell integrity. Microglial reaction was assessed using a rabbit polyclonal antibody to Iba-1 (1:1000; Wako, Neuss, Germany). To confirm that peripherally injected hMSC do not negatively affect the native brain cytoarchitecture, brain sections were stained with the histological dye Cresyl violet (1% w/v; Sigma Aldrich, St. Louis, Mo.).

Quantification of Cerebral Amyloid Plaque Load

To acquire images of the cortex and hippocampus used for quantifying Aβ amyloid plaque load, stained brain sections were imaged with an ×10 objective using a Zeiss Axiovert 200M/ApoTome microscope (Carl Zeiss, Germany) coupled with a Zeiss Axiocam HR camera (Carl Zeiss, Germany). For each of the 27 animals involved in this study, 10±2 stained brain sections with a thickness of 25 μm and spaced from each other by 24 slices were available for imaging. Among these, four sections were selected based on the following criteria: one section encompassing the cortex (between position AP 0.74 mm and AP 0.38 mm from Bregma), one section with the striatum (between the position AP −0.46 mm and AP −0.70 mm from Bregma), one section showing the dorsal hippocampus (between position AP −1.82 mm and AP −2.18 mm from Bregma) and one section encompassing the ventral hippocampus (between position AP −2.70 mm and AP −3.08 mm from Bregma). For each selected section, 1300×1030 contiguous images (with less than 5% overlapping) were captured for the cortex region (60±15 images per animal), and in the hippocampus region when present (13±7 images per animal). This generated 5619 images taken from both hemispheres and readily available for quantification of amyloid load, among them 2733 for the Thioflavin T-stained sections (2241 of the cortex and 492 of the hippocampus) and 2886 for the 6E10 stained sections (2386 of the cortex and 500 of the hippocampus). The imaged coronal brain sections (25 μm thickness) were analyzed with Image J. Area analyzed was adjusted manually so that each measurement was accurately measuring only the region of interest and adjusted to exclude brains regions other than cortex and hippocampus. Brain sections encompassing the cortex and hippocampus, stained for Thioflavin T or 6E10, were quantified for amyloid load. For the Thioflavin T analysis the image was inverted and then a threshold of approximately 180 (with a 5% variance to best adjust tissues for analysis) was applied in the default setting. For 6E10 analysis a threshold of 140 (with a 5% variance to best adjust tissues for analysis) was applied in the default setting. The number was determined by looking at multiple randomly selected tissues to find the best range that accurately displays the plaques without background noise from varying conditions. Thioflavin T-stained brain sections were analyzed with the parameters of larger than 5×5 pixels and with a circularity of 0.15. 6E10-stained brain sections were analyzed with the parameters of larger than 5×5 pixels and with a circularity of 0.07. Data were then processed using Statistica.

Quantification of Iba-1-Positive Immunoreactivity

To acquire images of the cortex and the hippocampus for quantifying microglia immunoreactivity, the imaging setup used was adjusted with the same parameters as for the amyloid plaque load quantification. For each animal, 10±2 stained brain sections with a thickness of 25 μm and spaced from each other by 24 slices were available for imaging. Among these, two sections were selected based on the following criteria: one section encompassing the cortex (between position AP 0.74 mm and AP 0.38 mm from Bregma) and one section showing the dorsal hippocampus (between position AP −1.82 mm and AP −2.18 mm from Bregma). The images were acquired as described in the previous section. In the end, 1610 images were acquired, among them 1370 of the cortex (38±3 images per animal) and 240 of the hippocampus (7±2 per animal). The coronal brain sections generated were then analysed with Image J. For each animal, all the 8-bit images were assembled into a stack in order to facilitate the processing. A threshold of 110 with a variance of 10% was applied to each stack. This threshold was selected in order to best display the microglia while minimizing the background noise. After visual inspection, regions of interest (ROI) were selected such that staining artefacts/dirt on the section was manually removed by cropping. The microglia were quantified using the analyze particles tool embedded in Image J. In order to consider both the single microglia and the microglial loads, the pixel size was not specified and the circularity parameter was set to 0.0-1.0. Data were then processed using Statistica.

Quantification of Cerebral Amyloid Angiopathy

To assess the effect of hMSC treatment on vascular AP amyloid deposition, the numbers of Thioflavin T-positive vessels was calculated in transgenic brain sections from treated aged and young APPPS1 animals. As positive control for vascular amyloid, we used brain sections from another APP transgenic mouse model that develops robust vascular amyloid pathology. Per animal, the numbers of Thioflavin T-positive vessels was calculated on four sections selected based on the following criteria: one section encompassing the cortex (between position AP 0.74 mm and AP 0.38 mm from Bregma), one section with the striatum (between the position AP −0.46 mm and AP −0.70 mm from Bregma), one section showing the dorsal hippocampus (between position AP −1.82 mm and AP −2.18 mm from Bregma) and one section encompassing the ventral hippocampus (between position AP −2.70 mm and AP −3.08 mm from Bregma). Data were then processed using Statistica.

Quantification of Hemosiderin-Positive Microhemorrhages

The numbers of microhemorrhages was evaluated following hMSC delivery in treated APPPS1 animals. As positive control, tissue sections were obtained from human liver with extensive iron deposition. Per animal, the numbers of Thioflavin T-positive vessels was calculated on four sections selected based on the following criteria: one section encompassing the cortex (between position AP 0.74 mm and AP 0.38 mm from Bregma), one section with the striatum (between the position AP −0.46 mm and AP −0.70 mm from Bregma), one section showing the dorsal hippocampus (between position AP −1.82 mm and AP −2.18 mm from Bregma) and one section encompassing the ventral hippocampus (between position AP −2.70 mm and AP −3.08 mm from Bregma). Data were then processed using Statistica.

hMSC Biodistribution Study

For the biodistribution study, amyloid-depositing aged (12.5 month-old) APPPS1 mice received a single intravenous delivery of hMSC (500,000 cells resuspended in 100 μl of LRS, n=5) and were analyzed after either 1 hour, 1 day or 1 week post-delivery. As control, age-matched APPPS1 mice received an intravenous injection of LRS (100 μl, n=5) and were analyzed after either 1 hour, 1 day or 1 week post-delivery. Another group of APPPS1 mice were injected intravenously with hMSC (same cell preparation as above, n=5) and, at the time of analysis (1 day, 1 hour or 1 week), received a transcardiac perfusion with ice-cold distilled water for ten minutes in order to remove all blood from the animal circulatory system. At the time of analysis, mice were euthanized by isoflurane inhalation and brain, liver, heart, lung, spleen and kidney were collected, snap frozen in liquid nitrogen and stored at −80° C. for biodistribution analysis. Tissues were first disrupted and homogenized in QIAZOL reagent (QIAGEN) and RNA was isolated from the aforementioned tissues using RNAeasy mini kit (QIAGEN) according to manufacturer's instructions. 1 μg of total RNA was used to synthesize cDNA (Qiagen RT kit). Quantitative RT-PCR was performed using SYBR green (Roche) in the Lightcycler 480 II (Roche). All mRNA expression levels were corrected for expression of the housekeeping gene cyclophilin. Primers sequences used were Human Alu I 5′-CGAGGCGGGTGGATCATGAGGT-3′ 5′-TCTGTCGCCCAGGCCGGACT-3′ as previously described (Prigent et al., 2014). In a subsequent experiment, we compared hMSC biodistribution after intravenous delivery (500,000 cells resuspended in 100 μl of LRS) at 1 hour in 12.5 month-old APPPS1 mice (n=5) as compared to age-matched wild-type mice (n=5). As a control, we also analyzed age-matched uninjected APPPS1 mice (n=4) as well as APPPS1 that received an intravenous delivery of LRS (100 μl, n=4). Procedure for biodistribution analysis was similar to that described previously.

Soluble Abeta Assessment by ELISA

Brain protein extracts and plasma were diluted to the fourth into sampling medium provided by the manufacturer and a final of 25 μl volume was loaded into a 96 well plates. Aβ42 measurements were performed according to the manufacturer's instructions (Peptide Panel 1 (6E10) Kit (1 Plate) V-PLEX™K15200E-1 Mesoscale Discovery, Gaithersburg). All levels of Aβ42 were normalized against total protein amount.

Western Blot Assessment of Aβ Degrading Enzymes

Brains were extracted and homogenized in RIPA buffer (10% weight/volume). Brain homogenates were centrifuged at 14,000 rpm for 15 min at 4° C. before the protein concentration was measured. The purified protein fractions were stored at −80° C. until western blotting. Total protein concentration of each sample was determined using a BCA protein assay kit (Pierce, Rockford, Ill., USA). For Western blot analysis, 25 μg of protein was loaded into a precast 15-well NuPAGE Novex 12% Bis-Tris gel (Invitrogen, Waltham, Mass.) for separation by electrophoresis and then transferred to a polyvinylidenedifluoride (PVDF) membrane as indicated in the manufacturer's instructions (GE Healthcare, UK). As primary antibodies, mouse monoclonal anti-β tubulin antibody (cell signaling), rabbit polyclonal antibody to Insulin degrading enzyme (IDE, Abcam ab32216), Neprilysin degrading enzyme (NPE-CD10, Santa Cruz, sc-46656) and Endothelin Converting Enzyme-1 (ECE-1, Abcam ab189843) were incubated at 4° C. overnight, followed by the appropriate secondary horseradish peroxidase antibody (Jackson Labs, Baltimore) incubation. The blots were visualized with BM chemiluminescence Western blot kit (Roche, Basel).

Cytokine Profiling

For cytokine measurements, total brain homogenates were centrifuged at 14,000 rpm for 15 min at 4° C. and supernatants were analyzed using the mouse pro-inflammatory panel 1 V-plex plate (K15048D-1 Mesoscale Discovery, Gaithersburg) according to the manufacturer's instructions and normalized against total protein content. The following cytokine were evaluated: Interferon-gamma (IFN□), Interleukin 1-beta (IL1□), Interleukin 5 (IL5), Interleukin 4 (IL4), Interleukin 2 (IL2), Interleukin 6 (IL6), Keratinocyte Chemoattractant/Growth-Regulated Oncogene (KC/GRO, also known as CXCL1), Interleukin 12 (IL-12p70 active heterodimer), Tumor Necrosis Factor alpha (TNFα), Interleukin 10 (IL-10).

Preparation of Soluble hMSC Factors for Intranasal Application

For the preparation of soluble hMSC factors, hMSC were cultured in atmosphere containing 5% oxygen. After reaching 80-100% confluency, cellular monolayers were washed once with Hanks'balanced salt solution to remove bovine serum. Serum-free cell-conditioned medium was harvested 56 hours later. The medium was concentrated ˜50 times, mixed with sucrose, and then preserved by vacuum foam drying technique. Two ml of water was added to a bottle containing dried sucrose-protein foam. A volume of 5 μl SCF was delivered in each mouse nostril for a total 10 μl intranasally per session. Young (3 month-old) Alzheimer APPPS1 mice received the repeated application of either SCF (n=6) or LRS as control (n=4). The animals were infused daily for a total experiment duration of 3 weeks. The animals were sacrificed 2 days after the last SCF application.

Characterization of Soluble Factors Secreted by hMSC

Vacuum dried sucrose-protein foam was dissolved in water for injections. Concentrations of solubilized proteins was measured by Raybiotech testing service. We have chosen Raybiotech Q2000 human cytokine array that detects 120 human inflammatory factors, growth factors, and chemokines: BLC, Eotaxin-1, Eotaxin-2, G-CSF, GM-CSF, I-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, BDNF, bFGF, BMP-4, BMP-5, BMP-7, betaNGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PLGF, SCF, SCF R, TGFalpha, TGF beta 1, TGF beta 3, VEGF-A, VEGFR2, VEGFR3, VEGF-D6Ckine, Ax1, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL29, IL-31, IP-10, I-TAC, LIF, Light, Lymphotactin, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, TSLP.

Results

Reduction of Cerebral Soluble Aβ Levels in APPPS1 Mice Following a Single Injection of hMSC

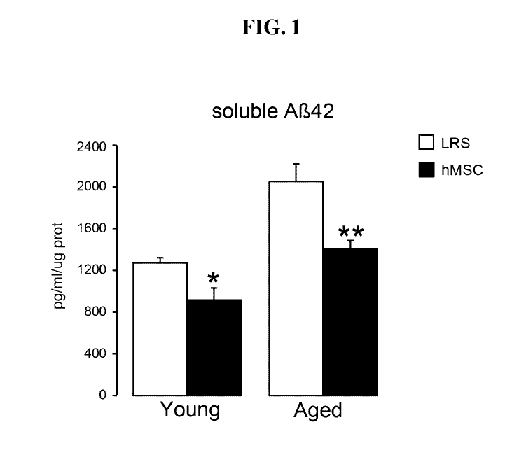

The Alzheimeric model used in this study is the APPPS1 mouse which co-expresses KM670/671NL mutated amyloid precursor protein and L166P mutated presenilin 1 under the control of a neuron-specific Thy1 promoter. Cerebral amyloidosis starts in these mice as early as 6-8 weeks of age and the number of Aβ amyloid plaques increases steadily. Single intravenous injections of hMSC were performed in APPPS1 mice and the levels of Aβ42 were analyzed by ELISA one week post-delivery. APPPS1 animals at both early stage of plaque development (i.e. young 3 month-old APPPS1 mice) and late stage (i.e. aged 12.5 month-old APPPS1 mice) were used. Control injections of LRS were performed in age-matched APPPS1 mice. Both young and aged APPPS1 hMSC-treated mice displayed reduced Aβ42 levels compared to the control mice (−28%, p<0.05 and −31%, p<0.01 respectively) (FIG. 1). These results indicate that a single injection of hMSC is sufficient to decrease soluble cerebral Aβ in both young and aged APPPS1 mice one week after injection.

Upregulation of Aβ-Degrading Enzymes in APPPS1 Mice After Single Injection of hMSC

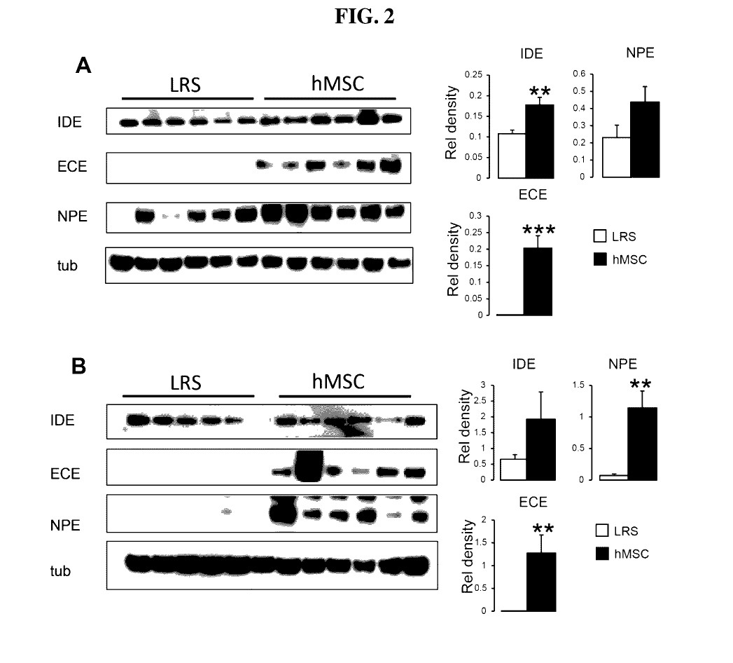

Several in vitro and in vivo studies have shown that Neprilysin degrading enzyme (NPE), Insulin degrading enzyme (IDE) and Endothelin-converting enzyme (ECE) can degrade Aβ (Farris et al., 2003; Apelt et al., 2003). We observed that NPE levels were increased in aged APPPS1 mice by 94% (p<0.01) (FIG. 2). Levels of IDE were increased in young APPPS1 mice by 40% (p<0.01), and similar in aged APPPS1 mice (p=0.18), as revealed by western blot analysis using an anti-IDE antibody (FIG. 2). As compared to age-matched APPPS1 mice, levels of ECE were increased in both young and aged APPPS1 animals by 99% (p<0.001 and p<0.01) (FIG. 2).

Decrease of Pro-Inflammatory Cytokines Following a Single hMSC Injection

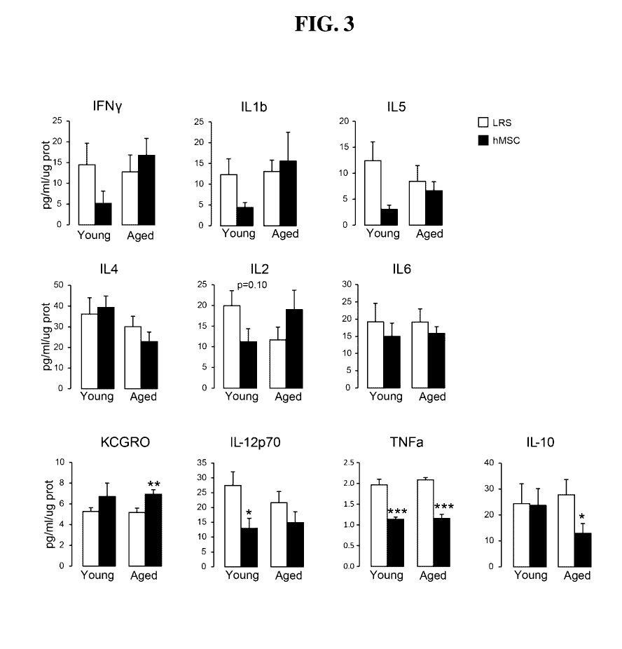

To further investigate the role of hMSC in Aβ pathology, we analyzed the impact of hMSC on cytokines levels. A diverse panel of cytokines was analyzed (IFNγ, IL1β, IL5, IL4, IL2, IL6, KCGRO, IL-12p70, TNFα, IL-10. In aged hMSC-treated mice animals, the levels of some pro-inflammatory cytokines such as IL-10 and IL-12p70 were both reduced by 54% (p<0.05) and by 53% (p<0.05), respectively. When compared to control mice at one week post-delivery, TNFα levels were decreased by 42% (p<0.001) and by 45% (p<0.001) in both young and in hMSC-treated mice aged animals respectively. (FIG. 3).

Reduction of Brain Aβ Immunoreactivity in Alzheimeric APPPS1 Mice Following Repeated hMSC Delivery

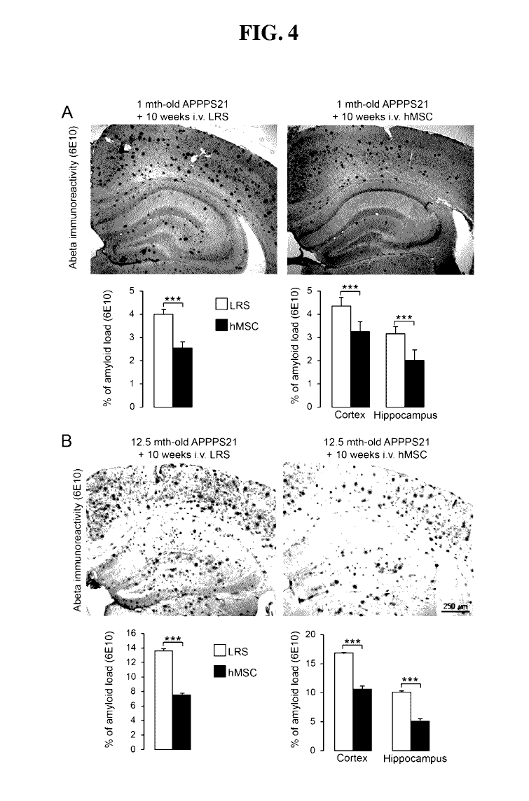

To determine whether a repeated administration of adult ischemia-tolerant hMSC reduces Aβ amyloid pathology in vivo, amyloid-depositing aged (12.5 month-old, n=7) and pre-depositing young (1 month-old, n=6) APPPS1 mice received a weekly intravenous hMSC injection for 10 weeks (FIG. 4). One injection consisted of a delivery through the tail vein of 500,000 cells in 100 μl of LRS. As controls, repeated injections of the same volume of LRS (100 μl) were performed weekly in aged (n=6) and young (n=8) APPPS1 mice (FIG. 4). One week after the last injection, the brains were collected and immmunostained with a mouse monoclonal anti-Aβ antibody (6E10) (see materials and methods for further details). This antibody was chosen to allow for the immunohistochemical detection of both types of amyloid plaques (diffuse and compact) in the brain of treated APPPS1 animals. Quantification of amyloid load on 6E10-stained sections revealed that cerebral Aβ amyloidosis was significantly reduced following HMSC treatment in aged (−44%, p<0.001) and young (−35%, p<0.001) APPPS1 animals, as compared to control aged-matched APPPS1 mice intravenously injected with LRS (FIG. 4). Both aged and young APPPS1 animals beneficiated from the hMSC delivery.

Decreased Congophilic Aβ Amyloidosis in hMSC-Treated APPPS1 Mice

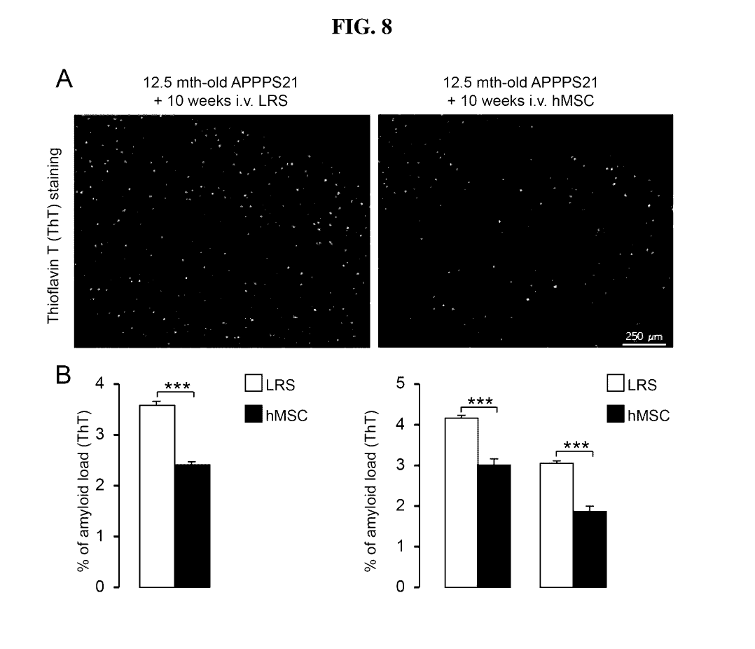

To investigate whether congophilic amyloid was lowered following repeated hMSC administration, brain sections were stained for congophilic amyloid (i.e., with Thioflavin T histological dye) in aged APPPS1 animals (FIG. 8). Quantification of compact amyloid load on Thioflavin T-stained brain sections revealed a significant reduction following hMSC treatment (−30%, p<0.001), as compared to control APPPS1 mice intravenously injected with LRS (FIG. 8). The impact of hMSC on reducing amyloid pathology was beneficial for both the cortex (−25%, p<0.001) and hippocampus (−36%, p<0.001) (FIG. 8). Altogether, these results demonstrate that not only diffuse but also congophilic amyloid in the Alzheimer mouse model studied is amenable [be more specific]to peripheral hMSC delivery.

Reduced Neuroinflammation in APPPS1 Mice Following hMSC Treatment

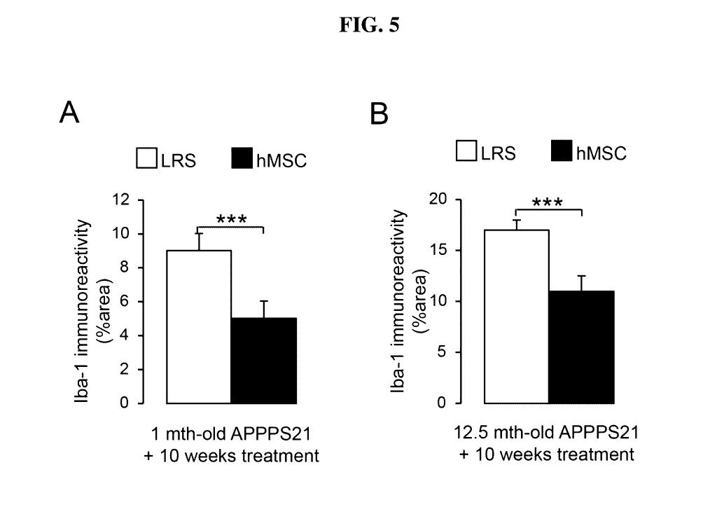

Microglial activation was examined to evaluate neuroinflammation changes in transgenic brains following repeated hMSC treatment. The reduction of amyloid load after a 10 weeks treatment was accompanied by an overall decrease in the microglial activation in brains of APPPS1 transgenic mice of both ages (FIG. 5). This qualitative observation was confirmed by quantitative image analysis of Iba-1 immunoreactivity, revealing a −35% and −39% in microglial immunoreactivity in aged and young APPPS1 mice, respectively (p<0.001 for both groups) (FIG. 5).

No Increase in Cerebral Amyloid Angiopathy Following hMSC Treatment

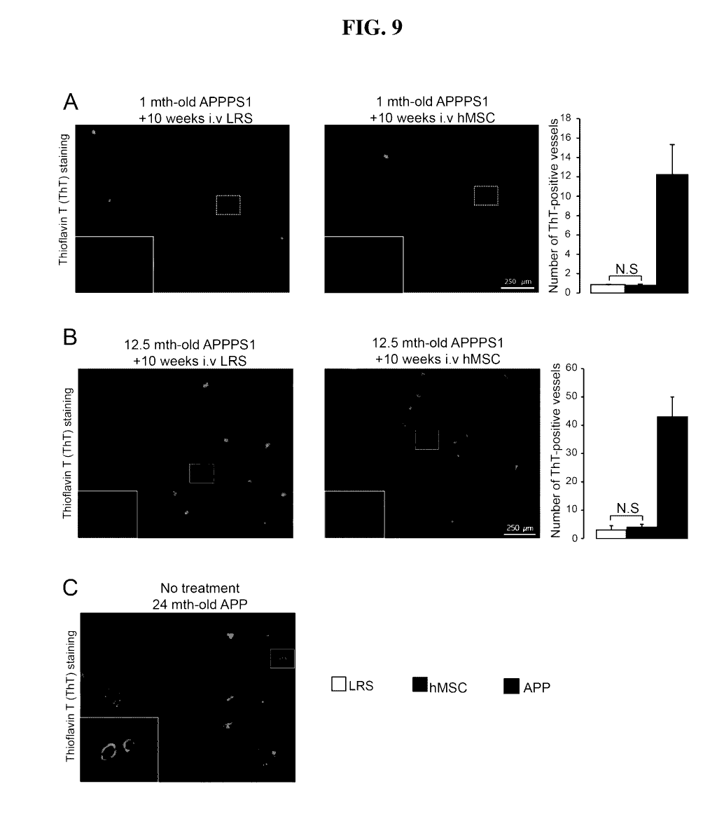

One reported side effect of the disruption of parenchymal amyloid plaques is increased cerebral amyloid angiopathy (CAA) (Pfeifer et al., 2002). To assess the effect of hMSC treatment on vascular Aβ amyloid deposition, the numbers of Thioflavin T-positive vessels has been calculated in transgenic brain sections from aged and young APPPS1 animals after the repeated hMSC treatments. No difference in the number of amyloid-containing blood vessels were observed in mouse brains injected with hMSC and control brains injected with LRS, in both aged and young APPPS1 mice (FIG. 9). As positive control, brain sections from an APP transgenic mouse model that develops robust vascular amyloid pathology was used. Even in aged APPPS1 animals, the numbers of Thioflavin T-positive vessels in control LRS-injected mice was very limited as the model normally develops rare cerebral amyloid angiopathy (Radde et al., 2006).

No Induction of Microhemorrhages Following Repeated hMSC Delivery

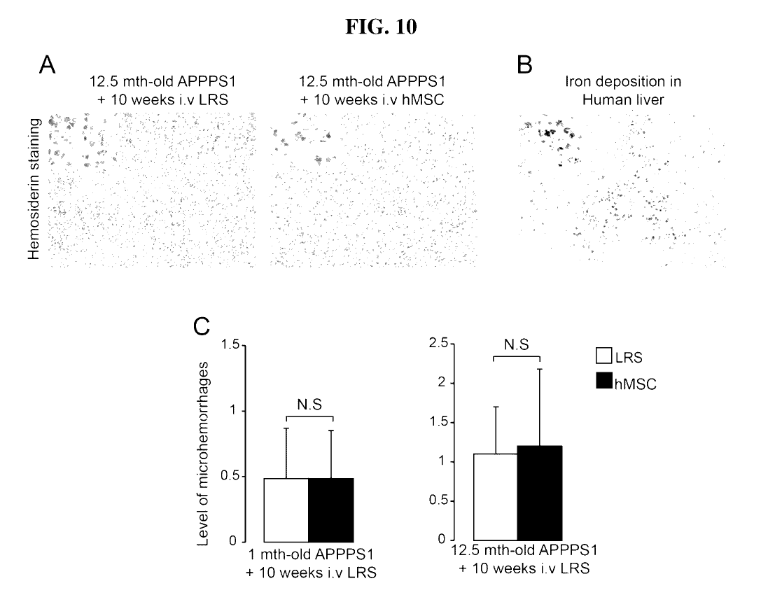

The numbers of microhemorrhages has been evaluated following intravenous hMSC delivery in aged and young APPPS1 animals (FIG. 10). As a positive control we used tissue sections from human liver with extensive iron deposition, which exhibits the characteristic blue hemosiderin-positive profile. However, repeated intravenous hMSC treatment did not induce microhemorrhages as compared to control LRS treatment. These results have been quantitatively confirmed. Safety of repeated hMSC injection was further supported by Cresyl violet coloration that failed to evidence any adverse effect on the cerebral architecture or the appearance of injury in any of the injected APPPS1 animals (data not shown). Safety of the repeated hMSC delivery was confirmed in aged non-transgenic animals (12.5 month-old, n=4) that received a weekly intravenous injection of hMSC for 10 weeks (data not shown).

Intravenously Delivered hMSC Migrate to the Mouse Brain

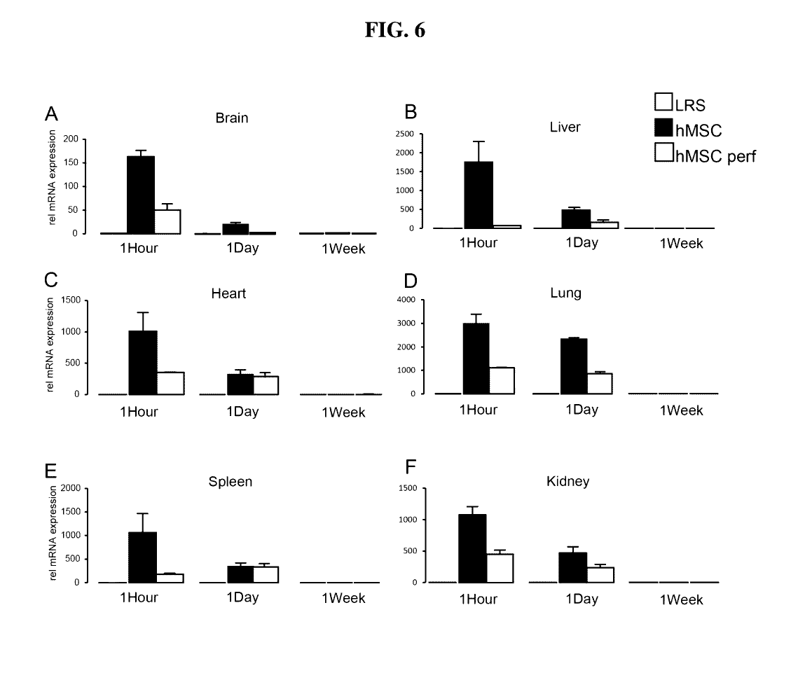

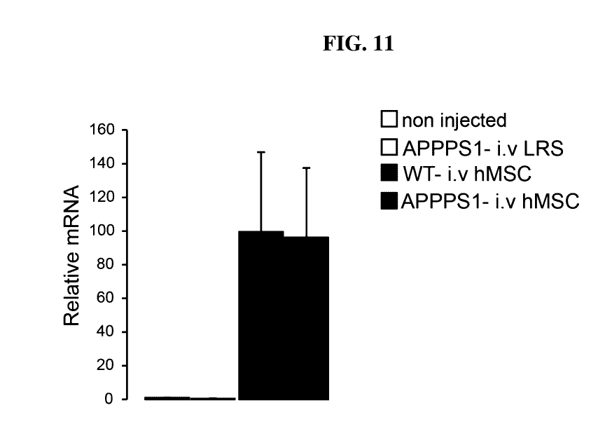

Biodistribution analysis after intravenous administration revealed that hMSC are readily detected in the brain of aged APPPS1 mice as early as 1 hour post-delivery (FIG. 6). The amount of hMSC detected in brains subsequently decreased by approximately 70% at 1 day post-delivery. The cerebral levels of hMSC reached negligible levels at 1 week post-delivery. Although hMSC were detectable by 1 hour post-delivery the amount of hMSC detected in the heart, spleen and kidney were approximately 7 times higher as compared to that quantified in the brain. The amount of hMSC detected in liver were approximately 10 times higher and in the lung 20 times higher in comparison to the brain. Comparison of hMSC biodistribution between perfused and non-perfused APPPS1 animals allowed to investigate the localization of hMSC for a given organ, i.e. whether the cells are located within the blood circulatory system or inside the organ's tissue. According to this model for the brain at 1 hour post-delivery, approximately 30% of hMSC relocated to the brain parenchyma and 70% were located within cerebral blood vessels. We next evaluated hMSC biodistribution in APPPS1 mice at 1 hour after intravenous delivery as compared to age-matched wild-type mice. Notably, the amount of intravenously-delivered hMSC was comparable between healthy and APPPS1 animals (FIG. 11).

### EXAMPLE 2

### Reduction of Aβ Amyloid Plaques in Alzheimeric APPPS1 Mice Following Repeated Intranasal Application of Soluble hMSC Factors

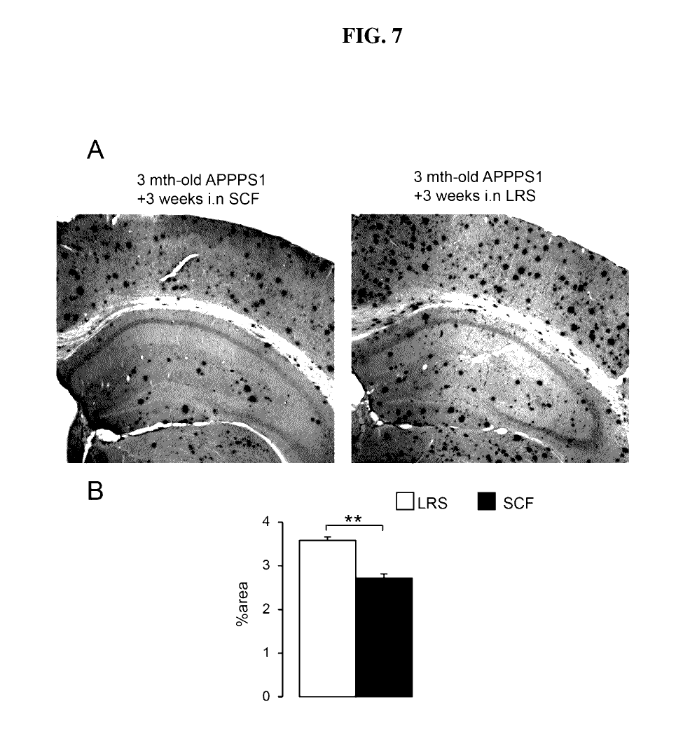

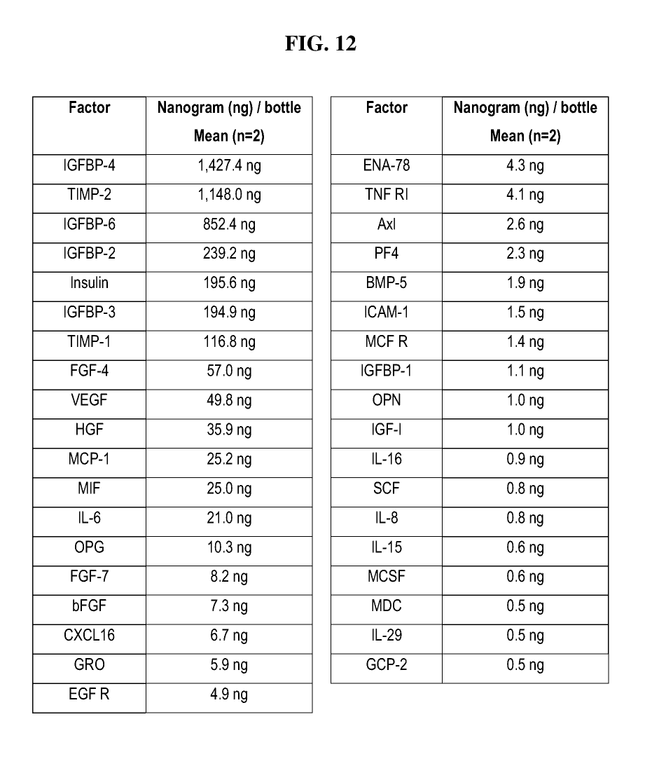

The following experiment was conducted to evaluate whether a peripheral application of a composition comprising soluble hMSC factors (SCF) in the absence of hMSCs, could positively impact cerebral Aβ amyloidosis in APPPS1 mice. To this end, hMSC were cultured at physiological level of 0₂ (5%), the cell-conditioned medium was collected, concentrated and preserved. SCF were subsequently reconstituted in LRS before intranasal application to APPPS1 animals. In a longitudinal study these factors were repeatedly administrated intranasally to APPPS1 mice. The protocol consisted of a daily intranasal delivery of SCF over a period of three weeks (intranasal delivery of 5 μl SCF solution/nostril/day). Two days after the last intranasal application, APPPS1 animals were sacrificed for histopathological analysis. Quantification of amyloid load on 6E10-stained sections revealed a significant decrease of Aβ plaques in the brain of APPPS1 mice following repeated intranasal application of SCF (FIG. 7), suggesting that these SCF may be used in combination hMSC or as a maintenance therapy after intravenous delivery of hMSCs. Identification and measurement of the concentration of SCF secreted by hMSC were performed with human cytokines, growth factors, as well as chemokines assays, and revealed the presence of many hMSC-secreted proteins including vascular endothelial growth factor-A (VEGF-A) (FIG. 12).

### REFERENCES

1. Apelt J, Ach K, Schliebs R. Aging-related down-regulation of neprilysin, a putative beta-amyloid-degrading enzyme, in transgenic Tg2576 Alzheimer-like mouse brain is accompanied by an astroglial upregulation in the vicinity of beta-amyloid plaques. Neurosci Lett. 2003 Mar. 27; 339(3):183-6. PubMed PMID: 12633883.

2. Balkwill F, Coussens L M. Cancer: an inflammatory link. Nature. 2004 Sep. 23; 431(7007):405-6. PubMed PMID: 15385993.

3. Banks W A, Terrell B, Farr S A, Robinson S M, Nonaka N, Morley J E. Passage of amyloid beta protein antibody across the blood-brain barrier in a mouse model of Alzheimer's disease. Peptides. 2002 December; 23(12):2223-6. PubMed PMID: 12535702.

4. Boado R J, Lu J Z, Hui E K, Sumbria R K, Pardridge W M. Pharmacokinetics and brain uptake in the rhesus monkey of a fusion protein of arylsulfatase a and a monoclonal antibody against the human insulin receptor. Biotechnol Bioeng. 2013 May; 110(5):1456-65. doi: 10.1002/bit.24795. Epub 2012 Dec. 25. PubMed PMID: 23192358; PubMed Central PMCID: PMC3919501.

5. Brookmeyer R, Johnson E, Ziegler-Graham K, Arrighi H M. Forecasting the global burden of Alzheimer's disease. Alzheimers Dement. 2007 July; 3(3):186-91. doi: 10.1016/j.jalz.2007.04.381. PubMed PMID: 19595937.

6. Bürger S, Noack M, Kirazov L P, Kirazov E P, Naydenov C L, Kouznetsova E, Yafai Y, Schliebs R. Vascular endothelial growth factor (VEGF) affects processing of amyloid precursor protein and beta-amyloidogenesis in brain slice cultures derived from transgenic Tg2576 mouse brain. Int J Dev Neurosci. 2009 October; 27(6):517-23. doi: 10.1016/j.ijdevneu.2009.06.011. Epub 2009 Jul. 7. PubMed PMID: 19589380.

7. Coussens L M, Werb Z. Inflammation and cancer. Nature. 2002 Dec. 19-26; 420(6917):860-7. Review. PubMed PMID: 12490959; PubMed Central PMCID: PMC2803035.

8. Fan X, Sun D, Tang X, Cai Y, Yin Z Q, Xu H. Stem-cell challenges in the treatment of Alzheimer's disease: a long way from bench to bedside. Med Res Rev. 2014 September; 34(5):957-78. doi: 10.1002/med.21309. Epub 2014 Feb. 5. PubMed PMID: 24500883.

9. Farris W, Mansourian S, Chang Y, Lindsley L, Eckman E A, Frosch M P, Eckman C B, Tanzi R E, Selkoe D J, Guenette S. Insulin-degrading enzyme regulates the levels of insulin, amyloid beta-protein, and the beta-amyloid precursor protein intracellular domain in vivo. Proc Natl Acad Sci USA. 2003 Apr. 1; 100(7):4162-7. Epub 2003 Mar. 12. PubMed PMID: 12634421; PubMed Central PMCID: PMC153065.

10. Hardy J, Allsop D. Amyloid deposition as the central event in the aetiology of Alzheimer's disease. Trends Pharmacol Sci. 1991 October; 12(10):383-8. Review. PubMed PMID: 1763432.

11. Hernández-Guillamon M, Delgado P, Ortega L, Pares M, Rosell A, García-Bonilla L, Fernández-Cadenas I, Borrell-Pagès M, Boada M, Montaner J. Neuronal TIMP-1 release accompanies astrocytic MMP-9 secretion and enhances astrocyte proliferation induced by beta-amyloid 25-35 fragment. J Neurosci Res. 2009 July; 87(9):2115-25. doi: 10.1002/jnr.22034. PubMed PMID: 19235898.

12. Johansson P, Åberg D, Johansson J O, Mattsson N, Hansson O, Ahrén B, Isgaard J, Åberg N D, Blennow K, Zetterberg H, Wallin A, Svensson J. Serum but not cerebrospinal fluid levels of insulin-like growth factor-I (IGF-I) and IGF-binding protein-3 (IGFBP-3) are increased in Alzheimer's disease. Psychoneuroendocrinology. 2013 September; 38(9):1729-37. doi: 10.1016/j.psyneuen.2013.02.006. Epub 2013 Mar. 6. PubMed PMID: 23473966.

13. Kim K S, Kim H S, Park J M, Kim H W, Park M K, Lee H S, Lim D S, Lee T H, Chopp M, Moon J. Long-term immunomodulatory effect of amniotic stem cells in an Alzheimer's disease model. Neurobiol Aging. 2013 October; 34(10):2408-20. doi: 10.1016/j.neurobiolaging.2013.03.029. Epub 2013 Apr. 24. PubMed PMID: 23623603.

14. Pfeifer M, Boncristiano S, Bondolfi L, Stalder A, Deller T, Staufenbiel M, Mathews P M, Jucker M. Cerebral hemorrhage after passive anti-Abeta immunotherapy. Science. 2002 Nov. 15; 298(5597):1379. PubMed PMID: 12434053.

15. Radde R, Bolmont T, Kaeser S A, Coomaraswamy J, Lindau D, Stoltze L, Calhoun M E, Jäggi F, Wolburg H, Gengler S, Haass C, Ghetti B, Czech C, Hölscher C, Mathews P M, Jucker M. Abeta42-driven cerebral amyloidosis in transgenic mice reveals early and robust pathology. EMBO Rep. 2006 September; 7(9):940-6. Epub 2006 Aug. 11. PubMed PMID: 16906128; PubMed Central PMCID: PMC1559665.

16. Religa P, Cao R, Religa D, Xue Y, Bogdanovic N, Westaway D, Marti H H, Winblad B, Cao Y. VEGF significantly restores impaired memory behavior in Alzheimer's mice by improvement of vascular survival. Sci Rep. 2013; 3:2053. doi: 10.1038/srep02053. PubMed PMID: 23792494; PubMed Central PMCID: PMC3690383.

17. Selkoe D J. The molecular pathology of Alzheimer's disease. Neuron. 1991 April; 6(4):487-98. Review. PubMed PMID: 1673054.

18. Spuch C, Antequera D, Portero A, Orive G, Hernández R M, Molina J A, Bermejo-Pareja F, Pedraz J L, Carro E. The effect of encapsulated VEGF-secreting cells on brain amyloid load and behavioral impairment in a mouse model of Alzheimer's disease. Biomaterials. 2010 July; 31(21):5608-18. doi: 10.1016/j.biomaterials.2010.03.042. Epub 2010 Apr. 28. PubMed PMID: 20430437.

19. Salem A M, Ahmed H H, Atta H M, Ghazy M A, Aglan H A. Potential of bone marrow mesenchymal stem cells in management of Alzheimer's disease in female rats. Cell Biol Int. 2014 December; 38(12):1367-83. doi: 10.1002/cbin.10331. Epub 2014 Jul. 17. PubMed PMID: 25044885.

20. Thirumangalakudi L, Samany P G, Owoso A, Wiskar B, Grammas P. Angiogenic proteins are expressed by brain blood vessels in Alzheimer's disease. J Alzheimers Dis. 2006 September; 10(1):111-8. Review. PubMed PMID: 16988487.

21. Tullis G E, Spears K, Kirk M D. Immunological barriers to stem cell therapy in the central nervous system. Stem Cells Int. 2014; 2014: 507905. doi: 10.1155/2014/507905. Epub 2014 Aug. 5. Review. PubMed PMID: 25165476; PubMed Central PMCID: PMC4138731.

22. Vertelov G, Kharazi L, Muralidhar M G, Sanati G, Tankovich T, Kharazi A. High targeted migration of human mesenchymal stem cells grown in hypoxia is associated with enhanced activation of RhoA. Stem Cell Res Ther. 2013 Jan. 7; 4(1):5. doi: 10.1186/scrt153. PubMed PMID: 23295150; PubMed Central PMCID: PMC3706803.

23. Wang P, Xie Z H, Guo Y J, Zhao C P, Jiang H, Song Y, Zhu Z Y, Lai C, Xu S L, Bi J Z. VEGF-induced angiogenesis ameliorates the memory impairment in APP transgenic mouse model of Alzheimer's disease. Biochem Biophys Res Commun. 2011 Aug. 5; 411(3):620-6. doi: 10.1016/j.bbrc.2011.07.003. Epub 2011 Jul. 13. PubMed PMID: 21771586.

24. Yan Y, Ma T, Gong K, Ao Q, Zhang X, Gong Y. Adipose-derived mesenchymal stem cell transplantation promotes adult neurogenesis in the brains of Alzheimer's disease mice. Neural Regen Res. 2014 Apr. 15; 9(8):798-805. doi: 10.4103/1673-5374.131596. PubMed PMID: 25206892; PubMed Central PMCID: PMC4146257.

25. Yun H M, Kim H S, Park K R, Shin J M, Kang A R, il Lee K, Song S, Kim Y B, Han S B, Chung H M, Hong J T. Placenta-derived mesenchymal stem cells improve memory dysfunction in an Aβ1-42-infused mouse model of Alzheimer's disease. Cell Death Dis. 2013 Dec. 12; 4:e958. doi: 10.1038/cddis.2013.490. PubMed PMID: 24336078; PubMed Central PMCID: PMC3877561.

1-97. (canceled)

98. A method for treating Alzheimer's disease in a subject in need thereof comprising administering to the subject a therapeutically effective amount of a composition comprising at least one stem cell factor.

99. The method of claim 98, wherein the at least one stem cell factor comprises one or more mesenchymal stem cell factors.

100. The method of claim 99, wherein the one or more mesenchymal stem cell factors are human mesenchymal stem cell factors.

101. The method of claim 99, wherein the one or more mesenchymal stem cell factors are obtained from mesenchymal stem cells cultured under low oxygen conditions.

102. The method of claim 99, wherein the one or more mesenchymal stem cell factors are obtained from mesenchymal stem cells cultured under low serum conditions.

103. The method of claim 99, wherein the one or more mesenchymal stem cell factors are obtained from mesenchymal stem cells from bone marrow, dermis, periosteum, synovium, peripheral blood, skin, hair root, muscle, uterine endometrium, adipose, placenta, menstrual discharge, chorionic villus, amniotic fluid and umbilical cord blood.

104. The method of claim 98, wherein the at least one stem cell factor comprises mesenchymal stem cell conditioned medium.

105. The method of claim 104, wherein the mesenchymal stem cell conditioned medium has been used to culture mesenchymal stem cells under low oxygen conditions.

106. The method of claim 98, wherein the at least one stem cell factor comprises recombinant stem cell factors.

107. The method of claim 98, wherein the composition is administered before and/or after the onset of amyloidosis in the subject.

108. The method of claim 98, wherein the subject has Alzheimer's disease.

109. The method of claim 98, wherein the composition is administered one or more times.

110. The method of claim 98, wherein the composition comprises lyophilized and/or vaporized stem cell factors.

111. The method of claim 110, wherein the composition comprises lyophilized and/or vaporized stem cell factors in a pharmaceutically acceptable carrier.

112. The method of claim 110, wherein the composition comprises at least one of chitosan and heparin.

113. The method of claim 98, wherein the at least one stem cell factor is selected from the group consisting of: BLC, Eotaxin-1, Eotaxin-2, G-CSF, GM-CSF, I-309, ICAM-1, IFN-gamma, IL-1 alpha, IL-1 beta, IL-1ra, IL-2, IL-4, IL-5, IL-6, IL-6sR, IL-7, IL-8, IL-10, IL-11, IL-12p40, IL-12p70, IL-13, IL-15, IL-16, IL-17, MCP-1, M-CSF, OPN, MIG, MIP-1-alpha, MIP-1 beta, MIP-1 delta, PDGF-BB, RANTES, TIMP-1, TIMP-2, TNFα, TNFbeta, sTNFRI, sTNFRII Amphiregulin, PF4, MCF R, BDNF, BMP-4, BMP-5, BMP-7, betaNGF, EGF, EGFR, EG-VEGF, bFGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, OPG, Insulin, IGF-I, M-CSF R, NGF R, NT-3, NT-4, Osteoprotegerin, PDGF-AA, PLGF, SCF, SCF R, MCSF, TGFalpha, TGF beta 1, TGF beta 3, VEGF-A, VEGFR2, VEGFR3, VEGF-D6Ckine, Ax1, BTC, CCL28, CTACK, CXCL16, ENA-78 (CXCL5), Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-1, MCP-2, MCP-3, MCP-4, MDC, MIF, MIP-3 alpha, MIP-3 beta, MPIF-1, MSP-alpha chain, NAP-2, Osteopontin, PARC, PF4, SDF-1 alpha, TARC, TECK, and TSLP.

114. The method of claim 113, wherein the at least one stem cell factor comprises ICAM-1, IL-6, IL-8, IL-15, IL-16, OPN, TIMP-1, TIMP-2, TNF RI, PF4, MCF R, BMP-5, EGF R, bFGF, FGF-4, FGF-7, HGF, IGFBP-1, IGFBP-2, IGFBP-3, IGFBP-4, IGFBP-6, OPG, Insulin, IGF-I, SCF, MCSF, VEGF, Ax1, CXCL16, ENA-78 (CXCL5), GRO, IL-29, MCP-1, MDC, MIF, and GCP-2.

115. The method of claim 98, wherein the method further comprises administering mesenchymal stem cells to the subject.

116. The method of claim 115, wherein the administered mesenchymal stem cells are expanded under low oxygen conditions prior to administration to the subject.

117. The method of claim 116, wherein the low oxygen conditions comprise culturing the administered mesenchymal stem cells in low serum.

118. The method of claim 115, wherein the composition is administered before and/or after the mesenchymal stem cells are administered to the subject.

119. The method of claim 115, wherein the administered mesenchymal stem cells are administered intranasally, intravenously, intrathecally, intraarterially, or intracranially.

120. The method of claim 115, wherein the composition is administered to the subject multiple times after the mesenchymal stem cells are administered to the subject.


SEQUENCE LISTING (2 sequences)

SEQ ID NO: 1 - DNA, 22 bp, Human
cgaggcgggtggatcatgaggt

SEQ ID NO: 2 - DNA, 20 bp, Human
tctgtcgcccaggccggact